(19) United States
(12) Patent Application Publication
(10) Pub. No.: US 2013/0011872 A1
(43) Pub. Date: Jan. 10, 2013

(54) STABLE ISOTOPIC BIOMARKER MEASUREMENT FOR THE DETECTION OF CANCER AND THE DETERMINATION OF EFFICACY OF TREATMENT IN DIAGNOSED CANCER PATIENTS

(76) Inventor: Philip M. Gabriel, Fort Collins, CO (US)

(21) Appl. No.: 13/538,359

(22) Filed: Jun. 29, 2012

Related U.S. Application Data
(60) Provisional application No. 61/504,546, filed on Jul. 5, 2011.

(51) Int. Cl. A61B 5/08; G01N 21/01; A61B 6/00; G01N 21/84

(52) U.S. Cl. 435/34; 435/288.7; 600/532; 600/473; 600/407

(57) ABSTRACT

The present invention relates to a method of detecting the presence of cancer cells in an animal such as a human or other mammal, comprising administering a measured volume of air to the subject wherein ¹⁶O₂ and ¹⁸O₂ are present and then measuring the δ¹⁸O in the subject's exhaled CO₂. A Photoacoustic Spectrometer system is used for measuring the amount of laser light (Infrared) absorbed by isotopes of CO₂(C¹⁶O₂, C¹⁸O₂) in the exhalant.

### RELATED APPLICATION

This application is a non-provisional application of commonly-assigned U.S. Provisional Application No. 61/504,546, filed Jul. 5, 2011, entitled STABLE ISOTOPIC BIOMARKER MEASUREMENT FOR THE DETECTION OF CANCER AND THE DETERMINATION OF EFFICACY OF TREATMENT IN DIAGNOSED CANCER PATIENTS, the entire disclosure of which is hereby incorporated by reference.

### FIELD OF THE INVENTION

The present invention relates to the use of trace gas analysis to detect the presence of cancer cells in a human or other mammal and to gas compositions and spectrometers for use in such analysis and to systems comprising such spectrometers. The present invention also relates to the use of trace gas analysis to measure δ¹⁸O in the CO₂ of exhaled breath, to use this measurement as an indicator of the presence and stage of cancer and to monitor the efficacy of post cancer treatments following diagnosis. The present invention also relates to a method of using a photoacoustic spectrometer with an optical cantilever transducer to measure such isotopic composition in exhaled breath.

### BACKGROUND OF THE INVENTION

### Significance

The quantitative analysis of exhaled breath can provide useful information about the health status of an animal (such as a human or other mammal). Compared with a direct measurement of the metabolites in blood samples, sampling and analysis of breath is preferable because it is non-invasive and the measurements are much simpler in the gas phase than in a complex biologic fluid, such as blood or urine. Mass spectrometry (MS) and gas chromatography mass spectrometry (GC-MS) instruments have identified more than 3000 unique substances in exhaled breath, including elemental gases such as nitric oxide (NO) and carbon monoxide (CO) and a multitude of volatile organic compounds [Berden, G. and Engeln, R., Cavity Ring-Down Spectroscopy, Techniques and Applications 2009: Wiley]. Measuring these and other trace constituents in exhaled breath provides a way of non-invasively monitoring inflammation, oxidative stress and other disease processes in the body. Such monitoring can be accomplished by measuring volatile organic compounds because certain diseases are indicated by a specific odor in exhaled breath, e.g. acetone>diabetes, carbonyl sulfide>OC-hepatic disease, etc. [Berden, G. and Engeln, R. 2009]. Several methods of breath analyses are presently in clinical use. For example, current progress in the development of a diagnostic test for lung cancer through the analysis of breath volatiles [Smith, D., \check{S}panêl, P. and Sulé-Suso, J., Advantages of Breath Testing for the Early Dagnosis of Lung Cancer. Expert Rev. Mol. Diagn, 2010. 10(3): p. 255-257] has been reviewed recently [P. J., Analysis of Volatile Organic Compounds in the Exhaled Breath for the Diagnosis of Lung Cancer. J. Thorac. Oncol, 2008. 3(7): p. 774-780; P. J., Progress in the Development of a Diagnostic Test for Lung Cancer through the Analysis of Breath Volatiles. J. Breath Res, 2008. 3(037014)]. The latter reference is noteworthy in that it provides an exhaustive review of breath tests used in the detection of different kinds of cancer. The breath tests critiqued include the use of volatile organic compounds, gases and isotopes. The present invention builds further on breath analysis but now with a novel approach, as disclosed herein, for detecting cancer based on measurement of stable oxygen isotopes (i.e. the δ¹⁸O in exhaled CO₂).

### Conventional Methods of Cancer Detection

The design of instruments to detect the presence of cancers has generally been based on expensive, invasive procedures such as exploratory surgery, CT Scans, PET Scans, or the use of radioactive tracers. As such, these procedures share the limitations of being invasive to the body (e.g. blood tests, exploratory surgery, laparoscopic biopsies, etc.), expensive (e.g. PET SCANS, MRI's, CT Scans, surgical procedure, etc.), potentially injurious to the patient (e.g. radioactive tracers, anesthetic procedures, surgery, etc.), and carry the risk of inadvertently spreading tumor cells (e.g. abdominal cavity surgery or any surgical procedures that expose cancerous tissues to exogenous air). In addition, radioactive biomarkers cannot be continually used on the same patient, nor should they be used in certain situations such as pregnancy, hyperthyroidism, etc. Due to the universal need for portability in cancer detecting instruments and the growing constraints on medical costs, the relatively large size, complexity and cost of the conventional instruments, quite apart from their operating deficiencies, limit their utility in medical practice. By contrast, the present invention employs no intrusive element, is relatively straightforward to implement and provides a measurement of the progression of treatment in patients previously diagnosed with cancer. Early stages of cancers may also be detected. The present invention can be used to routinely screen for cancers and monitor the efficacy of subsequent treatment, therefore it can be used by a variety of professionals in diverse domains; doctors, clinicians, research laboratories and cancer treatment centers (e.g. Memorial Sloan-Kettering Cancer Center, Mayo Clinic), hospitals, senior care centers, outpatient clinics, nursing homes and geriatric facilities, etc. Combined with its ease of distribution, the low (production and utilization) cost of the instrument will increase the ease of verifying the spread of cancer cells. Determining the efficacy of treatment will also significantly reduce the large expense of cancer diagnoses to the benefit of all strata of society.

### Use of Stable Isotopes

Breath biomarkers can be classified into two groups: a) breath components produced endogenously (inside the body) owing to a particular physiological status, and b) breath metabolites, such as ¹³CO₂, produced exogenously (outside the body), after administration of a labeled drug or substrate and generated in breath [Bergman, B. C., E. E. Wolfel, G. E. Butterfield, G. Lopaschuk, G. A. Casazza, M. A. Horning and G. A. Brooks, Active Muscle Whole-Body Lactate Kinetics After Endurance Training in Men. J. Appl. Physiol., 1999. 87: p. 1684-1696; Bergman, B. C., M. A. Horning and G. A. Casazza, E. E. Wolfel, G. E. Butterfield and G. A. Brooks, Endurance Training Increases Gluconeogenesis During Rest and Exercise in Men. Am. J. Physiol. Endocrinol. Metab., 2000. 278 (E244-E251)]. The use of isotopes as disease biomarkers is not new. Stable isotope ¹³C-labeled compounds have been widely used as diagnostic probes in research laboratories for over 30 years [Modak, A., Stable Isotope Breath Tests in Clinical Medicine: A Review. J. Breath Res., 2007. 1(014003).]. More recently, methods have been developed that use ¹³C (e.g. ¹³CO₂) as biomarkers for disease identification [Szulejko, J. E., M. McCulloch, .J. Jackson, D. L. McKee, J. C. Walker, T. Solouki., Evidence for Cancer Biomarkers in Exhaled Breath. Sensors Journal, IEEE, 2010. 10(1): p. 185-210]. An example of isotopic breath testing is demonstrated by the non-invasive verification of a helicobacter pylori infection in the gastro-intestinal tract by means of a ¹³CO₂ breath test employing laser spectroscopic detection [Berden, G. and Engeln, R. 2009]. While there is much speculation on the carcinogenic role of bacteria, definitive evidence exists [Uemura, N., S. Okamoto, S. Yamamoto, N. Matsumura, S. Yamaguchi, M. Yamakido, K. Taniyama, N. Sasaki, R. J. Schlemper, Helicobacter Pylori Infection and the Development of Gastric Cancer. N. Engl. J. Med, 2001. 345(11): p. 784-789] showing a predisposition to stomach cancer in Helicobacter pylori infected individuals. Chemical kinetics is common to both the preceding method and to the present invention. However, the present invention differs from the ¹³CO₂ method through the use of the stable isotope δ¹⁸O in exhaled CO₂ as a biomarker for cancer. This is advantageous because (1) rather than being localized in the stomach/intestines, the O₂ molecule is transported throughout the body by the blood thus making possible the detection of cancers systemically, (2) the amount of ¹⁸O₂/¹⁶O₂ by volume in inhaled air can be preset so to administer a controlled fixed sample of air; this method is unique to this invention (3) the instrument can measure the exhalant as a steady state signal in a predetermined time frame (4) the smallness of the O₂ molecule relative to the CO₂ molecule allows for increased diffusion across cellular membranes and (5) ¹⁸O₂ is safe to inhale (see http://www.isotope-cmr.com/index.php?page=oxygen).

### Laser Spectroscopic Breath Analysis

Laser-spectroscopic breath analysis is based on the absorption of laser light as the laser beam passes through the exhaled gas (laser absorption spectrometry). This attenuation, or reduction in the laser light intensity, is described by the Beer-Lambert Law which states that the absorption is exponentially attenuated as it travels through the gas. Attenuation by the gas occurs either at fixed wavelengths or over wavelength intervals. For the law to be valid, several conditions must be met: (1) the absorbers act independently, without scattering the light, (2) the beam must consist of parallel rays, traversing the same length in the absorbing medium, (3) the beam is at a single wavelength and (4) the intensity of the beam must not be of such intensity as to cause optical pumping, since this effect will deplete the lower energy levels and may give rise to stimulated emission. Breath analysis using laser spectroscopy involves tuning a laser to a known location in the spectrum where absorption by the exhaled gas(es) of interest is present. The concentration of the exhaled gas is related to the amount of light absorbed. Direct application of the Beer-Lambert Law to laser absorption spectrometry requires a measurement of a small change in the laser signal superimposed on a large background. Small signal levels of noise, introduced by the light source or the optical system, deteriorate the sensitivity of the technique. Practically, this sensitivity is limited to an absorbance of ˜10⁻³, far away from the shot noise (electronic noise) level, and is insufficient for this application. Different forms of spectroscopy, such as Intracavity Laser Absorption Spectroscopy [http://www.physics.ucf.edu/˜rep/conf_pubs/ConfPubs2011/ICLAS_SPIE2011.pdf.], Cavity Ring-Down Spectroscopy [LaFranchi, B. 2003. Cavity Ringdown Spectroscopy: History, Fundamentals and Applications. Available from: http://www.uvm.edu/˜jgoldber/courses/chem226/Lafranchi_CRDS.pdf], Frequency Modulated Absorption Spectroscopy [Silver, A. J., Frequency-modulation spectroscopy for trace gas species detection: theory and comparison among experimental methods. Applied Optics, 1992. 31(6): p. 707-717] and Photoacoustic Spectroscopy [Bageshwar, V. D., Pawar, S. A., Khanvilkar, V. V., Kadam, J. V., Photoacoustic Spectroscopy and Its Applications—A Tutorial Review, 2010. Eurasian J. Anal. Chem. 5(2): p. 187-203] have been developed to overcome the aforementioned difficulty and can be used to measure the attenuation and to calculate the concentration of the gas(es) of interest.

The first two forms of spectroscopy do not possess both the high sensitivity and the signal-to-noise levels required in the practice of the present invention. Laser Photoacoustic Spectroscopy has an advantage in that it is background free; it does not rely on a decrease of the transmitted light but on an increase from the zero baseline, i.e. on a collisional release of energy after absorption of laser energy by the gas. One embodiment of the present invention is realized by the use of Laser Photoacoustic Spectroscopy. In the practice of certain embodiments of the present invention, the gases of interest are stable oxygen isotopes, ¹⁸O₂ and ¹⁶O₂ occurring in exhaled breath for determining the δ¹⁸O. This quantity is used as a biomarker for the presence of cancer.

Laser-spectroscopic breath analysis offers the capability of online measurements for analyzing breath samples and displaying results within minutes on a monitor [Murtz, M., Breath Diagnostics Using Laser Spectroscopy. Optics and Photonics News, 2005. 16(1): p. 30-35]. In contrast, offline techniques, where the breath is collected and contained for later analysis, suffer from lack of reproducibility introduced by contamination during sample storage and the inability for immediate feedback. The methods of the present invention do not require breath collection and storage. Other advantages of laser breath analysis include the relatively low cost, versatility and portability.

### SUMMARY OF THE INVENTION

The present invention relates to a method for detecting the presence of cancer cells in an animal comprising administering a measured volume of gas comprising air or a mixture of gases wherein measured amounts of ¹⁶O₂ and ¹⁸O₂ are present and then measuring the δ¹⁸O in the CO₂ of the patient's exhaled breath. An increase of δ¹⁸O indicates the presence and stage of cancer. In one embodiment of the invention, the animal is a human or other mammal. In another embodiment of the invention, the administered rate of consumption of the air or a mixture of gases for a human is 5.92 to 14.54 liters/minute for individuals from birth to 81 years of age with the rates for higher ages to be determined by a physician based on the condition of the patient. In a particular embodiment, the rate is 5.92 to 14.54 liters/minute for a female from birth to 81 years of age and 6.08 to 14.54 liters/minute for a male from birth to 81 years of age in accordance with EPA (Environmental Protection Agency) regulation (Metabollically-Derived Human Ventilation Rates: A Revised Approach Based Upon Oxygen Consumption Rates, EPA/600-R-06/129F, May 2009)) with the rates for higher ages to be determined by a physician based on the condition of the patient.

In one embodiment of the invention, a premixed volume of three gases (¹⁶O₂ and ¹⁸O₂ and ¹⁴N₂) is applied in variable concentrations by volume. In a particular embodiment, the gaseous composition administered to a human (or other mammal) is represented by α(¹⁶O₂)+β(¹⁸O₂)+γ(¹⁴N₂) wherein α=10.5%, β=10.5% and γ=79% as well as to other gaseous combinations described herein.

In one embodiment of the invention, δ¹⁸O in exhaled CO₂ is determined by measurement of mid-infrared absorption using at least one mid-infrared laser source. In one embodiment, this is accomplished with a photoacoustic spectrometer detection system. The spectrometer systems and the methods for using such systems to detect the presence of cancer discussed in this Summary of the Invention are considered embodiments of the present invention.

In a particular embodiment, δ¹⁸O in exhaled CO₂ is determined by measuring mid-infrared absorption using a photoacoustic spectrometer detection system comprised of (1) two quantum cascade lasers, (2) two modulators, (3) two photoacoustic cells, (4) two cantilever acoustic transducers, (5) two mirrors, (6) two beam splitters, (7) two Michelson-type interferometers, (8) computer, (9) 4 channel analog-to-digital converters, (10) software to control the modulators, to convert the cantilever movements to concentrations of ¹⁶O and ¹⁸O and to calculate δ¹⁸O and (11) a visual display device. In a particular embodiment, the system comprises a sample cell and a reference cell and each modulator is a device for periodically blocking the light emitted by said laser and each beam splitter directs the intermittent laser light to the reference cell and also redirects the intermittent laser light onto a mirror which illuminates the sample cell.

The present invention also relates to a method for detecting cancer from changes in cell membrane permeability of cells transitioning to the cancerous state comprising administering a measured volume of air wherein ¹⁶O₂ and ¹⁸O₂ are present and then measuring the δ¹⁸O in the patient's exhaled CO₂ wherein the value of δ¹⁸O in exhaled carbon dioxide indicates the presence and stage of cancerous cells in the body.

The cancers detected can be selected from a wide spectrum of types that include, but are not limited to lung, breast, ovarian, colon, prostate and pancreatic.

In a particular embodiment, the gas concentrations in the patient's exhaled CO₂ flowing through the photoacoustic spectrometer system are measured simultaneously at wave numbers 2312.85 and 2313.15 cm⁻¹. In another embodiment, the fractional sensitivity of the photoacoustic spectrometer, defined by:

$$δ {}^{18} {O}_{sample 1} - δ {}^{16} {O}_{sample 2} = 1000 ( \frac{{{(}^{ } {}^{18} O {/}^{ } {}^{16} O )}_{sample 1} - {( {}^{18} O / {}^{16} O )}_{sample 2}}{{( {}^{18} O / {}^{16} O )}_{VSMOW}} ) .$$

is, in terms of the minimum discernible changes in δ¹⁸O, at least 8.3×10⁻². The denominator in the above expression, (¹⁸O/¹⁶O)_{VSMOW} is a fixed, known standard quantity referred as the “Vienna Standard Mean Ocean Water”. Thus the spectrometer can resolve differences in δ¹⁸O in the exhalate as small as 0.083. This value is limited by the signal to noise ratio that is set by the power of the laser and properties of the cantilever transducer, as described in section “SENSITIVITY OF THE PHOTOACOUSTIC SPECTROMETER WITH OPTICAL CANTILEVER DETECTOR”. Alternatively, the fractional sensitivity is equivalent to the minimum detectable difference of two samples of exhalate in the ratio ¹⁸O₂/¹⁶O₂, divided by (¹⁸O/¹⁶O)_{VSMOW}, in which case the fractional sensitivity is 8.3×10⁻⁵.

In a particular embodiment, the patient's exhaled CO₂ is analyzed by the photoacoustic spectrometer system to obtain δ¹⁸O, Said δ¹⁸O is visually displayed on an appropriate visual device, and the presence and stage of cancer cells is indicated as increasing differences in the δ¹⁸O averages between populations of healthy individuals and individuals afflicted with cancer. In a particular embodiment, the pressure of the gas in the photoacoustic spectrometer system is maintained at 8 kPa.

The present invention also relates to a photoacoustic spectrometer system for detecting the presence of cancer cells in an animal, including but not limited to humans and other mammals, comprising means for administering a measured volume of gas comprising air or a mixture of gases wherein measured amounts of ¹⁶O₂ and ¹⁸O₂ are present and means for measuring the δ¹⁸O in the CO₂ of the patient's exhaled breath. In a particular embodiment, the system comprises a breathing apparatus for human patients or for other mammals, wherein the breathing apparatus for humans consists of a mask connected to a flow system and the breathing apparatus for other mammals consists of a tent connected to a flow system.

In one embodiment of the invention, the gas concentrations in the patient's exhaled CO₂ flowing through the photoacoustic spectrometer system can be measured simultaneously at wave numbers 2312.85 and 2313.15 cm⁻¹. In another embodiment of the system, the fractional sensitivity of the photoacoustic spectrometer, in terms of the minimum discernible changes in δ¹⁸O, at least 8.3×10⁻².

In another embodiment, the system of the present invention comprises means for analyzing the animal's exhaled CO₂ to obtain δ¹⁸O and, after the Receiver Operating Characteristic analysis, results are displayed on an appropriate visual device, so that the presence and stage of cancer cells is indicated as increasing differences in the δ¹⁸O averages between populations of healthy individuals and individuals afflicted with cancer. Another embodiment of the system of the present invention comprises means for maintaining the pressure of the gas in the photoacoustic spectrometer system within the range 7.7-8.3 kPa with 8 kPa as the preferred value.

The present invention also relates to combinations of the foregoing embodiments.

### BRIEF DESCRIPTION OF THE DRAWINGS

FIG. 1 shows an absorption spectrum produced by a spectral simulation of CO₂ lines. Isotopes are labeled in the figure. Simulation conditions are P=8 kPa, T=295 K, [CO₂]=390 ppm with natural isotopic abundances. Isotopologues are labeled in the figure.

FIG. 2 shows stages whereby an acoustic signal is generated in a gas at an absorbing wavelength.

FIG. 3 shows a side view of an optical cantilever pressure sensor. Typical dimensions are given.

FIG. 4 shows a top view of an optical cantilever pressure sensor. Typical dimensions are given.

FIG. 5 shows principal elements of a photoacoustic system to measure ¹⁸O₂/¹⁶O₂ in CO₂ of exhaled human breath.

FIG. 6 shows principal elements of a photoacoustic system to measure ¹⁸O₂/¹⁶O₂ in CO₂ in the exhalate of mammals.

FIG. 7 shows examples only of the histograms of normal subjects and subjects afflicted with cancer at some stage of development. The threshold, δ_{T}=−2.5 indicated is used, among other thresholds in the calculation of the receiver operating characteristic curve.

FIG. 8 is similar to FIG. 7, except δ_{T}=−5.1.

FIG. 9 shows the receiver operating characteristic curve computed using different values of δ_{T}.

### DETAILED DESCRIPTION OF THE INVENTION

### Spectral Signatures of Exhaled Isotopologues of CO₂

### (1) Selected Spectral Interval

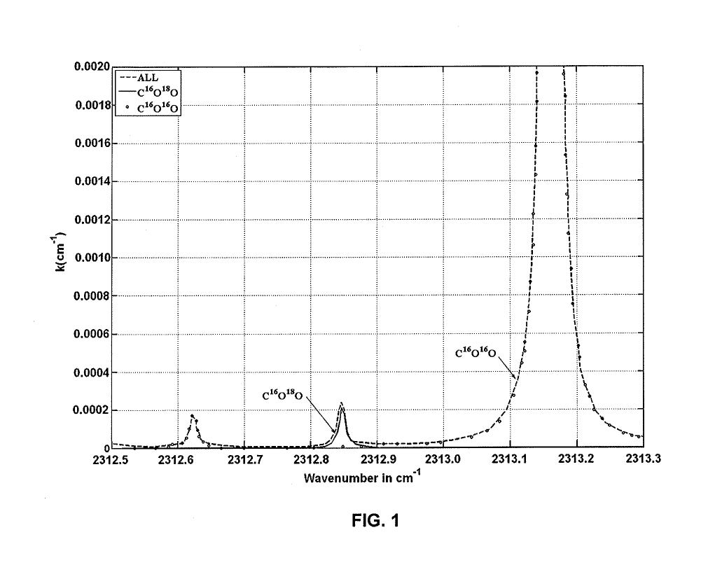

Spectral simulations have been performed to identify wavelength regions within which the instrument used to detect the presence and stage of cancer operates. The photoacoustic laser spectrometer of the present invention uses a spectral interval in the mid-infrared spectrum (i.e. 4.3 or 2309 cm⁻¹) to probe the spectral lines of the targeted isotopologues of carbon dioxide, ¹²C¹⁸O¹⁶O and ¹²C¹⁶O¹⁶O in the exhalant. The latter isotopologue is also written as ¹²C¹⁶O₂. Isotopologues of a molecule share the same molecular structure, but their individual atoms are different isotopes. Any sample of atmospheric air will contain a mixture of CO₂ isotopologues in fixed proportions. For example, 98.4% of the CO₂ molecules will be the primary isotopologue, ¹²C¹⁶O, while ¹²C¹⁸O¹⁶O will account for 0.39%. The remaining fraction is made up of more common and less common isotopologues (e.g. ¹³C¹⁶O₂ and ¹²C¹⁶O¹⁷O respectively) whose line strengths are orders of magnitude weaker than those shown in FIG. 1, hence would be difficult to detect. These fixed percentages are sometimes referred to as natural or terrestrial isotopic abundances. Spectral data (i.e. line strengths and positions) obtained from the extremely precise, high-resolution transmission molecular absorption database (HITRAN) [Rothman, L. S., et. al., The HITRAN 2008 molecular spectroscopic database. Journal of Quantitative Spectroscopy & Radiative Transfer, 2009. 110(9-10): p. 533-572] were used in standard line shape methods to produce the absorption spectra shown in FIG. 1. This spectral database consists of measured and computed spectra of many different gasses and their isotopologues. Mid-infrared lasers are tuned so they overlap with the absorption lines at 2312.85 and 2313.15 cm⁻¹ shown in FIG. 1. All HITRAN species in this spectral region were included as a means to examine interferences. The curve indicated by the broken line includes the contributions from the nine isotopologues of carbon dioxide in the aforementioned wavenumber interval. These include, in order of decreasing abundance: ¹²C¹⁶O¹⁶O, ¹³C¹⁶O¹⁶O, ¹²C¹⁶O¹⁸O, ¹²C¹⁶O¹⁷O, ¹³C¹⁶O¹⁸O, ¹³C¹⁶O¹⁷O, ¹²C¹⁸O¹⁸O, ¹²C¹⁷O¹⁸O, and ¹³C¹⁸O¹⁸O. FIG. 1 shows that lines from the different CO₂ isotopes are clearly distinguishable and individually measurable while interferences from other common molecules such as ¹⁸O, ¹⁶O, H₂O, CO, and other organic volatile gases present in exhaled breath are absent or negligible. It is also seen that contributions by the abundant ¹²C¹⁶O¹⁶O are virtually nil, while contamination by other isotopologues of carbon dioxide are practically, insignificant. The system of the present invention comprises pressure regulating device for maintaining the pressure of the gas in the photoacoustic spectrometer system at 8 kPa. A pressure at 8 kPa was found to be a reasonable tradeoff between line strengths (which decrease with decreasing pressure) and interferences (lines become sharper and isolated with decreasing pressure) The system of the present invention also comprises means for analyzing the animal's (human's or other mammal's) exhaled CO₂ to obtain δ¹⁸O and a method for visually displaying said δ¹⁸O on an appropriate visual device, so that the presence and stage of cancer cells is indicated as increasing differences in the δ¹⁸O averages between populations of healthy individuals and individuals afflicted with cancer.

### (2) Detection of ¹²C¹⁶O₂ and ¹²C¹⁸O¹⁶O in the Exhalant by Photoacoustic Spectroscopy

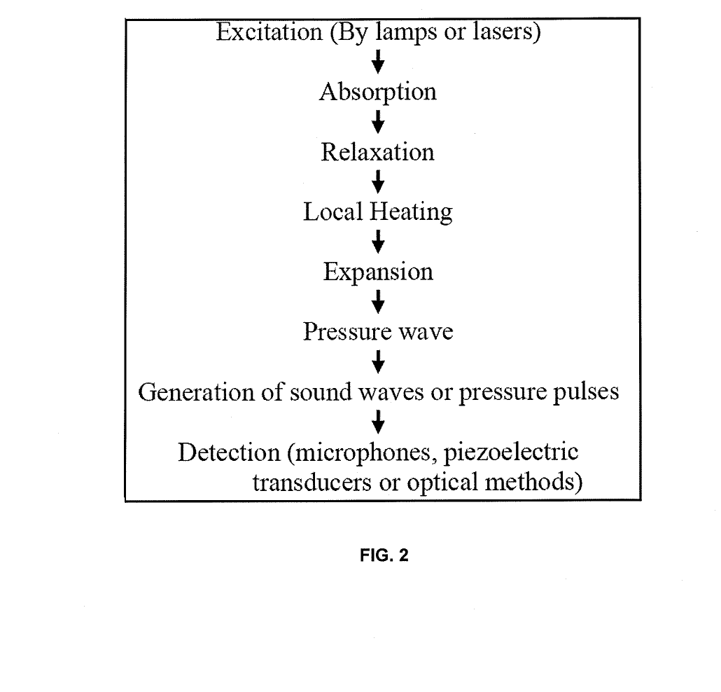

In photoacoustic spectroscopy, periodic modulation of a source of radiant energy such as a laser, results in the generation of thermal energy within the sample exhibiting the same period as the source. The resulting (acoustic) thermal pressure propagates through the volume of gas to a detector such as an electret microphone, quartz crystal or a transducer that utilizes optical methods, to produce an electrical signal. The amplitude of this signal is a function of the wavelength of the laser and yields a spectrum that is proportional to the absorption spectrum of the exhalant isotopic composition of CO₂. The method of detection has an advantage in that the magnitude of the detected signal is proportional to the absorbed laser power and that the sensor (i.e., the microphone) itself has a wavelength-independent responsivity. Photoacoustic spectroscopy can be applied to substances in solid, liquid or gas phases. As used in the methods, systems and spectrometers of the present invention, this form of spectroscopy utilizes the absorption of electromagnetic radiation by the gaseous molecules ¹²C¹⁸O¹⁶O and ¹²C¹⁶O₂ in exhaled CO₂. The photoacoustic signal in which an optical event is transformed into an acoustical event, is the result of two types of processes; absorption of electromagnetic radiation and thermal propagation in the sample. The stages of this transformation are summarized in FIG. 2.

Laser source options in the mid-infrared region, include gas lasers (CO, CO₂), lead-salt diode lasers, coherent sources based on difference frequency generation techniques, optical parametric oscillators, quantum and inter-band cascade lasers. Pulsed quantum cascade and inter-band cascade lasers are commonly employed in photoacoustic spectroscopy since they exhibit narrowband output, high-power and a broad tuning range (exceeding 100 inverse centimeters) and can be operated at room temperatures. However, owing to the high pulse intensities in the laser pulse, absorption by molecular gases can enter into a non-linear regime that is characterized by multiphoton absorption and saturation. Such nonlinearity can cause pulse-to-pulse signal fluctuations, thereby reducing detection sensitivity. This effect is considered in the calculation of the signal-to-noise ratio mentioned later in this section. According to [Harren, F. J. M., Mandon, J., Cristescu, M. S. Photoacoustic Spectroscopy in Trace Gas Monitoring. Encyclopedia of Analytical Chemistry (2012). DOI10.1002/9780470027318.a0718.pub2], modulated continuous wave lasers offer more sensitive detection (up to a factor of a thousand), compared to pulsed lasers. However, due to the unavailability of gas lasers operating in the desired spectral interval, the quantum cascade laser will be used to probe the isotopologues in the exhalant. Modulation of the laser can be accomplished either mechanically, with a rotating chopper wheel, or electronically, by pulsing the laser source. The modulated laser beam excites the gaseous sample inside a (usually cylindrical) photoacoustic cell resulting in a sound wave that is detected using microphones. The high spectral brightness of laser sources makes them ideally suitable for photoacoustic trace gas detection. In contrast to direct absorption techniques, the photoacoustic signal is proportional to the laser power. From the Beer-Lambert Law, for small absorption, the law takes the form: P=P₀ exp(−σNl)≈P₀(1+σNl), where P₀ is the laser power incident on the photoacoustic cell and P the power transmitted through the photoacoustic cell; σ the absorption cross-section per molecule (cm²), N the number of absorbing molecules per cubic centimeter, and/the absorption path length (cm). The absorbed power ΔP=(P₀−P) is converted into acoustic signal recorded by an acoustic transducer. As can be seen from this equation, for small absorption, the generated acoustical signal is proportional to the incoming laser power (high laser powers are advantageous) and the gas concentration (linearity of the signal). A nonlinear absorption response occurs only in focused high-power laser beams as a result of saturation. Lasers can achieve very high selectivity because their line widths can be made narrower than those of the absorbing gas. In general, the highest sensitivities of photoacoustic detection are achieved with modulated continuous wave lasers.

To detect trace gases, an ideal photoacoustic cell should amplify the generated sound originating from the molecular gas absorption and reject any acoustic (and electric) noise and in-phase infrared absorption from other substances. This necessitates the use of selective amplifiers to lower ambient acoustic and electric noise levels, thus improving the signal-to-noise ratio. Other requirements for photoacoustic cells are low gas consumption and a fast response. For this, the active volume of the cell should be small so that no dilution can take place when the trace gas and its carrier flow through the acoustic cell.

In the methods, spectrometers and systems of the present invention, a nonresonant, cylindrical cell characterized by the absence of standing waves is used owing to the extremely sensitive and fragile nature of the optical cantilever pressure sensor described in Section 3. The performance of a nonresonant cell can be expressed as the efficiency to convert absorbed photon energy into acoustic energy, i.e. p_{gas}=FΔP, with p_{gas} the generated acoustic pressure in the gas and ΔP [Harren, F. J. M., Mandon, J., Cristescu, M. S. Photoacoustic Spectroscopy in Trace Gas Monitoring. Encyclopedia of Analytical Chemistry (2012). OI10.1002/9780470027318.a0718.pub2].

The nonresonant cell constant Fₙᵣ (in units of Pa cm W⁻¹) is from the expression:

$${F}_{nr} = \frac{G ( γ - 1 ) l}{ω V}$$

where l and V are the length and volume of the cell, respectively, γ the specific heat constant, ω, the modulation frequency (radians sec⁻¹), and G a geometrical factor of order unity. From the above formula, one can derive that Fₙᵣ is independent of the cell length. The diameter should be as small as possible to generate high signals, just large enough to allow proper alignment of the incident laser beam. To minimize the effects of heat generation caused by the absorption of laser energy by the cell windows, it is necessary to maximize the cell length because the energy absorbed by the windows is then distributed over a larger volume. Since the photoacoustic signal decreases with higher modulation frequencies it is advantageous to use low modulation frequencies.

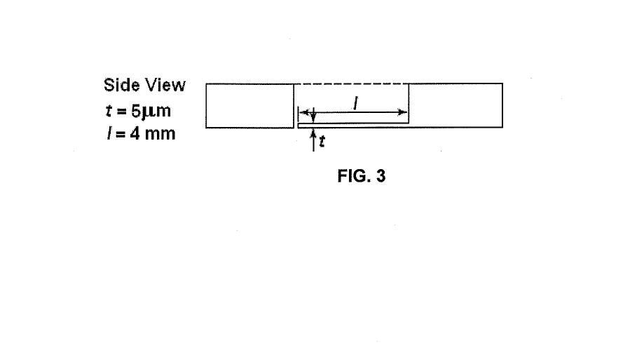

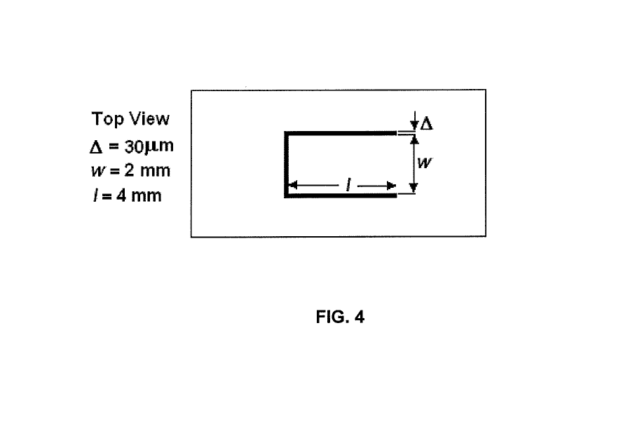

To realize the high degree of sensitivity required for the measurements of δ¹⁸O described herein, the photoacoustic spectrometer employing the aforementioned cantilever type pressure sensor described by [Uotila, J., Use of the Optical Cantilever Microphone in Photoacoustic Spectroscopy. TURUN YLIOPISTON JULKAISUJA ANNALES UNIVESITATIS TUKUENSIS, SARJA—SER.AI—TOM 395, ASTRONOMICA—CHEMICA—PHYSICA—MATHEMATICA, 2008. ISBN 978-951-29-3912-1] has been adopted. The cantilever, made out of silicon using a micofabrication process based on two-sided etching of silicon-on-insulator wafers as shown in FIG. 3 and FIG. 4, moves like a double acting cafe door whose motion is induced by a pressure difference over the cantilever. This cantilever design is employed in the photoacoustic spectrometer manufactured by Gasera Ltd. (http://www.gasera.fi/products/ftir-accessories/pa101/).

The cantilever is typically 5 μm-10 μm thick and is supported by much thicker (300 μm-500 μm) frames. A narrow micrometer sized gap separates the cantilever and its frame on three sides as shown in FIG. 4. The main benefit of the cantilever is its extremely wide dynamic range of movement, which can be tens of micrometers without any non-linear or restricting effects. An interferometric measurement of the cantilever end movement is required to avoid damping friction caused by other means such as probes and to maintain its wide dynamic range of movement. More detailed information about the properties of the cantilever design may be found in [Uotila, J. 2008].

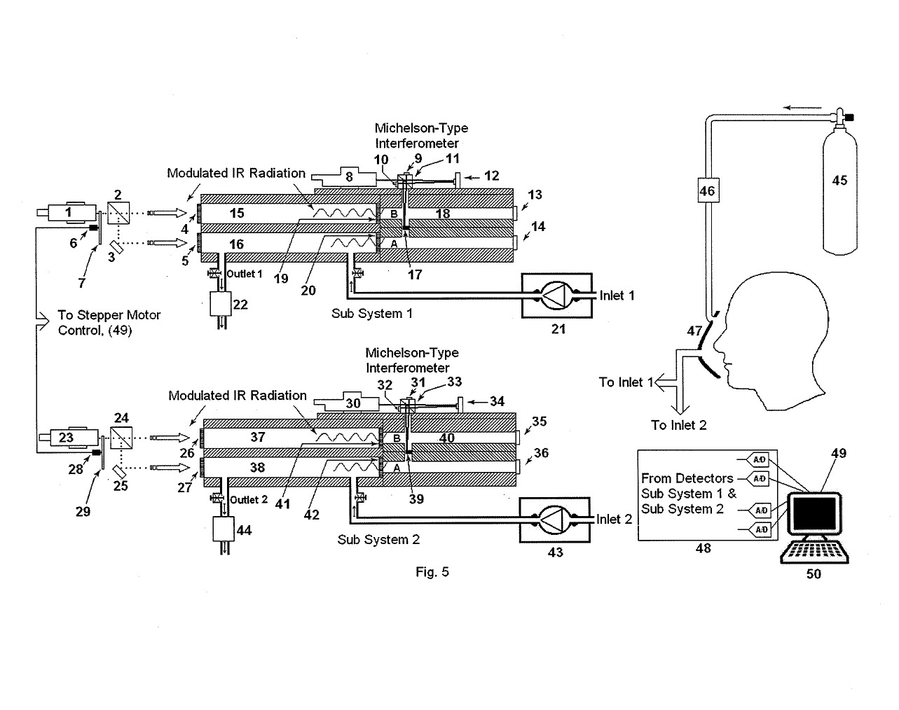

In order that the invention be better understood, an embodiment thereof will be described by way of example only with reference to the accompanying drawings. In FIG. 5, the photoacoustic spectrometer system is comprised of two subsystems labeled Sub System 1 and Sub System 2 that function independently to measure the concentrations of ¹²C¹⁶O¹⁶O and ¹²C¹⁸O¹⁶O in the exhalate. As such, it is only necessary to understand a single subsystem (e.g. Sub System 1) since its principles of operation also apply to Sub System 2. Furthermore, in this particular embodiment, two QCL lasers operating at 2312.85 cm⁻¹ 2313.15 cm⁻¹ and respectively, (i.e. components 1 and 23) are employed to simplify the exposition. Use of a single laser would require beam steering as well as a laser source capable of being swept between 2312.85 cm⁻¹ and 2313.15 cm⁻¹ and additional synchronization circuitry.

A QCL laser tuned to 2312.85 cm⁻¹ (component 1) is modulated by a rotating chopper wheel (component 7) attached to a stepper motor (component 6) controlled by a stepper motor control card (component 49) inserted into a pci slot of the computer mother board. The beam splitter (component 2) in front of the QCL laser (component 1) divides the periodically interrupted beam into two orthogonal beams of equal power. The direct beam exits the beamsplitter undeflected and is directed to an infrared window (component 4) that is transparent to the laser radiation, where it enters a reference cell (component 15) filled with nitrogen at one atmosphere of pressure. Nitrogen is used because it does not absorb the laser radiation, is commercially readily available and is inexpensive. The beam propagates through the reference cell and penetrates a second transmissive window (component 19) into region B of the photoacoustic cell. This region (component 18) is filled with pure ¹²C¹⁸O¹⁶O at one atmosphere of pressure. Here the beam periodically heats the gas which is strongly absorbing at 2312.85 cm⁻¹ and induces an acoustic pressure wave that is designated as P_{b}. To increase the efficiency of the heating, hence the generation of sound, a portion of the laser signal is reflected back into this cavity by a mirror (component 13) attached to the right side of the photoacoustic cell.

The component of the laser beam diverted 90 degrees with respect to the direct beam by the beam splitter is reflected by a mirror (component 3) to an infrared window (component 5) attached to the sample cell. The laser radiation upon entry, is partially absorbed by the ¹²C¹⁸O¹⁶O in the human exhalate. The pressure in this cell is maintained at about 8 kPa by a pressure regulator device. The laser radiation enters region A of the photoacoustic cell through an infrared window (component 20) where it induces a pressure wave designated as Pₐ. The efficiency of sound generation is increased by a mirror (component 14) that reflects back into this region the laser radiation.

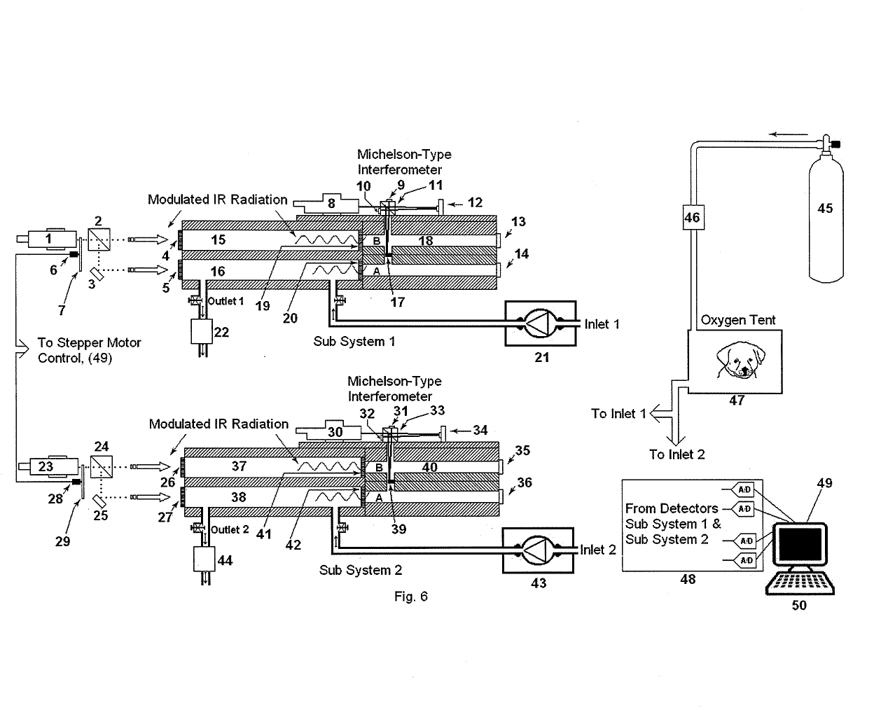

The pressure wave, Pₐ-P_{b} across the cantilever transducer (component 17) causes it to oscillate. The oscillation amplitude is detected by the Michelson interferometer. The latter consists of a Helium-Neon laser (component 8) operating at 632.8 nm, a beam splitter (component 11), mirror (component 12) and two detectors (components 9 and 10). The outputs of the detectors are analog signals that are directed to a four-channel digital-to-analog converter (component 48). The digitized signals are then used by a computer (component 50) to synthesize the amplitude of the pressure wave from which the concentration of ¹²C¹⁸O¹⁶O is calculated as described by [Uotila, J. 2008]. Thus a photoacoustic cell is seen to act as a highly selective detector. The isotopologues ¹²C¹⁸O¹⁶O and ¹²C¹⁸O¹⁶O are the result of metabolic processes, described later in this document. The ¹⁸O¹⁶O and ¹⁶O¹⁶O isotopes, present in the exhaled carbon dioxide are preset in the air the patient inhales (component 45). This preset air mixture may be custom ordered from ICON SERVICES in Summit, N.J. Analyzing human exhalant requires a mask (component 47), an adjustable flow regulator (component 46) as required by the EPA and a premixed air source having a high ¹⁸O₂/¹⁶O₂ ratio (e.g. a 1:1 mixture) that the patient inhales. The exhaled breath is equally divided and directed to subsystem 1 and to subsystem 2 for analysis. An airflow control system (components 21 and 22) maintains the pressure in the sample cell (component 16) at the preferred value of 8 kPa. Hence δ¹⁸O whose definition is given in the section: “DETECTION OF THE PRESENCE OF CANCER IN EXHALANT USING PHOTOACOUSTIC SPECTROSCOPY”, can be calculated by measuring concentrations of ¹²C¹⁸O¹⁶O and ¹²C¹⁸O¹⁶O gases in the exhalant simultaneously at the wave numbers 2312.85 cm⁻¹ and 2313.15 cm⁻¹ flowing through subsystem 1 and subsystem 2. The components of subsystem 2 map exactly onto those of subsystem 1 except that the QCL laser (component 23) operates at 2313.15 cm⁻¹ and the infrared windows (components 26,27,41,42) must be transparent at these wavenumbers. It is understood that the mirrors must all be highly reflective at the wavenumbers employed. Finally software is required to control the modulators, to convert the cantilever movements measured by the Michelson-type interferometer to concentrations of ¹⁶O and ¹⁸O, hence calculate δ¹⁸O. Component 50 includes a visual display device connected to the computer for displaying the results. For veterinary applications, the premixed gas enters an oxygen tent and the exhaled breath of the animal is directed equally through subsystem 1 and subsystem 2 as shown in FIG. 6. The principle of operation of these two subsystems is identical to the ones used to analyze human exhalate, with the exception that the EPA regulations applied to human subjects regarding oxygen consumption rates are not applicable. Hence the components of the photoacoustic system are the same as those shown in FIG. 5.

In one embodiment of the invention, the fractional sensitivity of the photoacoustic system, in terms of the minimum discernible changes in δ¹⁸O, is at least 8.3×10⁻². The present invention permits an immediate feedback of the efficacy of cancer treatments, performed routinely without any detrimental effects to the subject. Owing to changes in cell membrane permeability of cells transitioning to the cancerous state and isotope substitution, the δ¹⁸O in the exhaled CO₂ contained in breath relates to the presence of cancerous cells in the body. The presence and stage of cancer cells is indicated as increasing differences in the average δ¹⁸O between populations of healthy individuals and individuals afflicted with cancer.

Different types of cancer will cause the δ¹⁸O signal to exhibit different signal-to-noise ratios. The signal-to-noise ratio of the δ¹⁸O signal can be increased by changing the volume concentrations of the inhaled gaseous isotopes, expressed by the composition: α(¹⁶O₂)+β(¹⁸O₂)+γ(¹⁴N₂) such that α+β+γ=1 and 0.21≦α+β≦0.235. The second constraint not only allows for changing the concentration of oxygen isotopes, but is also imposed to eliminate the toxic effects of enhanced oxygen levels and the threat of fire in closed spaces. This constraint is compliant with OSHA requirements for human subjects. Furthermore, in accordance with EPA standards, the system in the present invention comprises means for controlling the rate of air consumption in a range of 6.08 to 14.54 liters/minute for a human male from newborn to age 81 and in a range of 5.92 to 11.25 liters/minute for a human female from newborn to age 81 [EPA/600-R-06/129F, May 2009] and for older patients as directed by a physician. The coefficients α, β, (hence γ) are determined empirically according to the type and stage of the cancer. In one embodiment of this patent, the gaseous composition administered to a human (or other mammal) is: α=10.5%, β=10.5% and γ=79%. The selection of these values is the same as the volume concentrations in the atmosphere (i.e. 21% ¹⁶O₂ and 79% N₂). The difference is that the ¹⁸O₂ concentration is greatly elevated.

Sensitivity of the Photoacoustic Spectrometer with Optical Cantilever Detector

Good results were achieved in several studies using the optical cantilever detector. At best, a noise equivalent absorption coefficient of 1.7×10⁻¹⁰ cm⁻¹ W/\sqrt{Hz}, close to the shot noise limit for their cell has been reported [Uotila, J. 2008]. As shown in FIG. 1, the absorption lines to be probed have peak absorptions of 0.025 cm⁻¹ (major isotope—¹²C¹⁶O₂) and 2.1×10⁻⁴ cm⁻¹ (¹⁸O isotope—¹²C¹⁸O¹⁶O). The probe laser to be used (distributed feedback QCL from Alpes Laser) has 10 mW of power. Owing to possible laser linewidth effects and the duty cycle for scanning an individual line (versus other line and baseline), an effective laser power of 1 mW has been adopted, providing a noise equivalent sensitivity of 1.7×10⁻⁷ cm⁻¹/\sqrt{Hz}. Combining the line strengths with the sensitivity of the photoacoustic system yields predicted signal-to-noise ratios of SNR_{Major}=1.5×10⁶ and SNR_{18O}=1.2×10⁴ for the major isotope and ¹⁸O isotope respectively, for 100-s measurement times. Since SNR_{Major}>>SNR_{18O}, the limit of detection is set by the heavier isotopologue and is (SNR_{18O})⁻¹ which yields a fractional sensitivity, in terms of the minimum discernible changes in δ¹⁸O, of at least 8.3×10⁻². Alternatively, the fractional sensitivity is equivalent to the minimum detectable difference of two samples of exhalate in the ratio ¹⁸O₂/¹⁶O₂, divided by the “Vienna Standard Mean Ocean Water” standard reference, (¹⁸O/¹⁶O)_{VSMOW}, in which case the fractional sensitivity is 8.3×10⁻⁵. This sensitivity compares favorably with Aerodyne Inc's. TILDAS sensor (see http://www.aerodyne.com/sites/default/files/NASAInstru2009.pdf). Another advantage of this design is that the lower states of the measured lines of both isotopes originate from the vibrational ground state, weakening the temperature dependences (fractional intensity change of <0.006/K for both lines, as opposed to 0.02/K for the major isotope of the Aerodyne sensor). This should lessen the temperature and flow control precision requirements.

The basis of the present invention rests on the relationship between the isotopic content of premixed inhaled air and the exhalant. The spectrum plotted in FIG. 1 assumes the subject inhales ambient air consisting of roughly 80% nitrogen and 20% oxygen. Two isotopologes of oxygen dominate the oxygen component of air: (¹⁸O₂ and ¹⁸O₂), available from ICON SERVICES, Summit, N.J. By premixing the oxygen isotopologue composition so that the rarer isotope ¹⁸O₂ is more abundant relative to atmospheric air, the signal-to-noise ratio of the aforementioned isotopologues in the exhaled CO₂ will increase. The present invention develops this unique concept, which has never been implemented in biomedical research and is a brand new development in the detection of cancer. The mechanisms are described in the next section. The methodology is not limited to the detection of cancers in humans, but is also applicable to all areas of veterinary medicine for mammals.

### Detection of the Presence of Cancer in Exhalant Using Photoacoustic Spectroscopy

Chemical reactions in the mitochondria take place in an aqueous environment. Oxygen isotopes present in the water can substitute themselves for the oxygen contained in certain compounds involved in the Krebs Cycle. The CO₂ released in the process of aerobic respiration will contain a certain ratio of ¹⁸O₂/¹⁶O₂. Isotope ratios usually are expressed in terms of the δ (“del”) notation in per mille (parts per thousand, ‰) relative to a standard material. For the case of ¹⁸O₂/¹⁶O₂, the corresponding δ is given by:

$$δ {}^{18} O = ( \frac{{}^{18} O / {}^{16} O}{{( {}^{18} O / {}^{16} O )}_{S}} - 1 ) 1000$$

where the denominator is a constant known as the “Vienna Standard Mean Ocean Water”, having a value of (2005±0.45)×10⁻⁶ [Emsley, J., “Oxygen”. Nature's Building Blocks: An A-Z Guide to the Elements 2001: Oxford University Press]. During the respiration process, the δ¹⁸O can be measured in exhaled CO₂. Catalyzed by carbonic anhydrase, the oxygen atoms of CO₂ and those of the body H₂O are rapidly exchanged in the final stage of the Krebs Cycle; the electron transport chain [Obuchowski, N. A., Receiver Operating Characteristic Curves and their Use In Radiology, 2003 Radiology, 229, 3-8]. At this point, inhaled oxygen is converted to water, which can take up either the heavy or light isotope of oxygen. The δ¹⁸O of the CO₂ is reflected in a steady-state balance of δ¹⁸O of the body H₂O.

This balance is caused by the various forms of intake and output of oxygen atoms such as the δ¹⁸O of inhaled O₂ in air, drinking H₂O, oxygen of food, etc. This uptake is counter-balanced by the δ¹⁸O loss from evaporation, CO₂ and H₂O loss by respiration and loss of oxygen by other natural processes. Table 1 presents data gathered from a small human population study of healthy individuals [Epstein, S. and Zeiri, L., Oxygen and Carbon Isotopic Compositions of Gases Respired by Humans. Proc. Natl. Acad. Sci. USA, 1988. 85: p. 1727-1731]. The relevance of this table is that it shows that the magnitude and variation of exhaled δ¹⁸O and defines (initial) requirements for the sensitivity of the photoacoustic spectrometer (i.e. spread of ˜3‰ leading to a minimum sensitivity requirement of 0.3‰). Variations in the δ¹⁸O of the body H₂O span a range of about 3.1‰, reflecting diet, drinking habits, and oxygen-isotope fractionation during respiration.

TABLE 1
δ¹⁸O values of respired CO₂ in a study of healthy humans.

| | Respired O₂ | Respired CO₂ |
|---|---|---|
| Participant | z ‰ | ¹⁸O ‰ |
| 1 | 13.0 | — |
| 2 | 12.2 | −5.4 |
| 3 | 12.2 | −6.8 |
| 4 | 11.6 | −5.6 |
| 5 | 11.0 | −5.4 |
| 6 | 11.0 | — |
| 7 | 10.8 | — |
| 8 | 10.5 | −4.3 |
| 9 | 10.0 | −7.4 |
| 10 | 9.6 | −6.1 |
| 11 | 9.6 | −6.4 |
| 12 | 9.6 | −4.9 |
| 13 | 9.4 | −4.9 |
| 14 | 9.2 | −5.7 |

The presence of neoplasms will cause departures from the measured norm. The normal population, i.e. one that is deemed cancer free, will exhibit a density distribution of the number of individuals exhibiting a particular value of δ¹⁸O in the breath test. Similarly, the population diagnosed as being afflicted with cancer at some stage of development will also exhibit a characteristic density distribution. In this discussion, and only for the sake of exposition, the normal and afflicted populations have been ascribed continuous, Gaussian distributions. The density of afflicted populations is given by 0.5exp(−(δ+4)²); normal populations by 0.5exp(−(δ+5.7)²). By density, is meant the number of subjects exhibiting a value of δ that falls within an, ‘infinitesimal’ bin, since the distributions are continuous. In practice histograms would be used for finite populations, in which case the bin sizes would be prescribed.

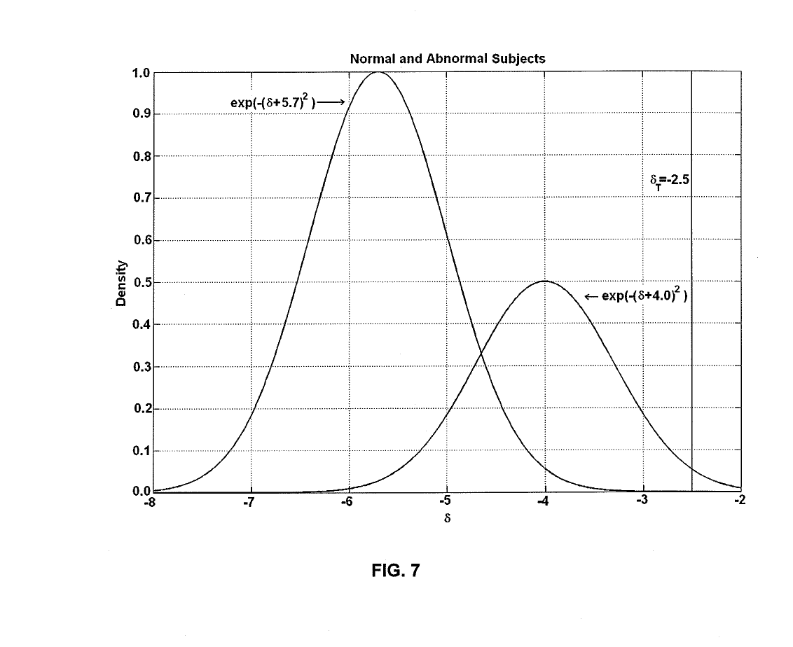

In the biomedical field and in veterinary medicine, interest is focused on how well a test can pick out subjects with a disease, (sensitivity) and the ability of a test to pick out subjects who do not have the disease (specificity). Sensitivity and specificity are the basic measures of the accuracy of a diagnostic test. The accuracy of a test is evaluated by comparing the results of the test to the true disease status of the subject as determined by standard procedures such as biopsies and/or follow-ups. The present invention does not focus in the predictive probability that a subject has cancer, but rather on a test (e.g. the use of δ¹⁸O) to detect cancer in its various stages and a test threshold (e.g. δ_{T} as shown in FIGS. 7,8) that allows accurate detection of the disease. Employment of receiver operating characteristic (ROC) curves, which assess the discriminatory power of diagnostic tests in correctly classifying diseased and nondiseased subjects, are designed to address such questions [Obuchowski, N. A., Receiver Operating Characteristic Curves and their Use In Radiology, 2003 Radiology, 229, 3-8] and will be used here. Such curves provide a measure of accuracy by combining sensitivity and specificity in a way that does not depend on the prevalence of disease.

Metrics can be developed to determine the health status of a subject, based on the four possible outcomes of a test. Erroneous test results are classified as either false positives (FP) or false negatives (FN). The former occurs when the test indicates the subject has cancer, when in fact no cancer is present. The latter indicates the absence of cancer, when in fact, cancer is present. In the absence of test errors, the remaining two indications are that the subject does have cancer, i.e. a true positive (TP) result, or does not have cancer, i.e. a true negative (TN) result. The test used to determine if a subject has cancer developing at a certain stage, requires first plotting the ROC curve and then applying a threshold, δ_{T}. The test indicates the presence of cancer if the subject's δ¹⁸O≧δ_{T}. This curve is readily plotted using the normal and afflicted populations by calculating the sensitivity and specificity defined as:

$$Sensitivity = \frac{TP}{TP + FN}$$

$$Specificity = \frac{TN}{TN + FP}$$

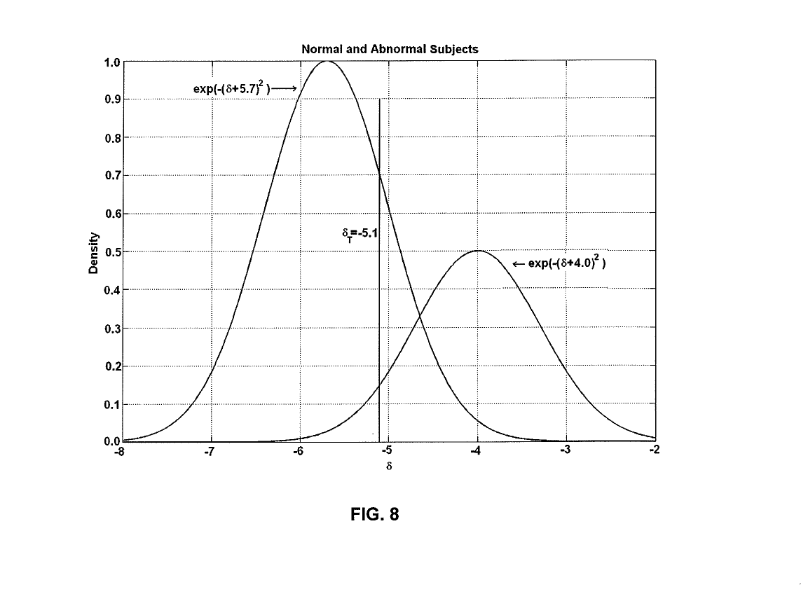

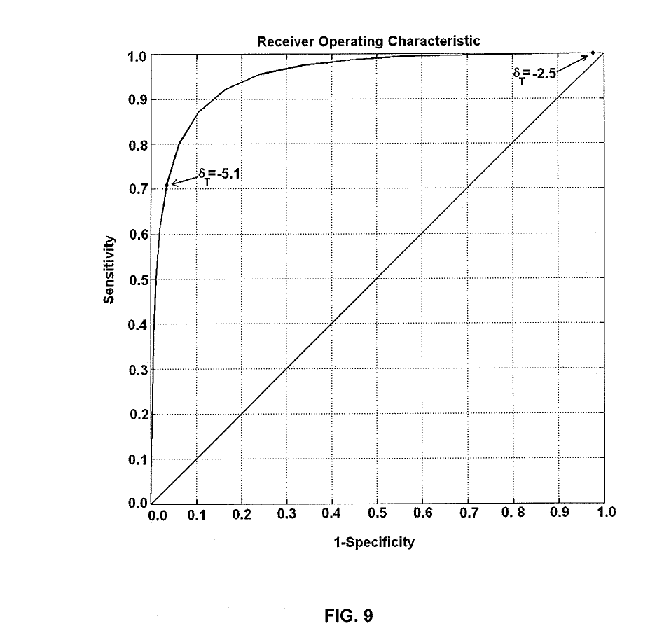

For example, in FIG. 7, the sensitivity is equal to the area under the curve (i.e. that of the afflicted population distribution) to the right of δ_{T}=−2.5 divided by its total area, which equals the sum of the true positive and false negative cases. Specificity is equal to the area under the curve (i.e. that of the normal population distribution) to the left of δ_{T} divided by its total area, which in this case, equals the sum of the true negative and false positive cases. The abscissa and ordinates associated with point δ_{T}=−2.5 are (1-specificity, sensitivity), as shown in the ROC curve in FIG. 9. Performing the same calculations at δ_{T}=−5.1 shown in FIG. 8 yields the coordinates marked for that threshold on the ROC curve. Repeating the calculation over a range of δ_{T} values completes the entire ROC curve shown in FIG. 9. The question of determining an optimal threshold has been answered [Zweig, M. H., Campbell, G. Receiver-operating characteristic (ROC) plots: a fundamental evaluation tool in clinical medicine, 1993. Clin. Chem., 39, 561-577] by finding the sensitivity and specificity pair that maximizes the objective function: sensitivity−m(1−specificity), where m is the slope of the ROC curve given by:

$$m = \frac{{Prob}_{Norm}}{{Prob}_{Dis}} \frac{( {C}_{FP} - {C}_{TN} )}{( {C}_{FN} - {C}_{TP} )}$$

where Prob_{Norm} is the probability that the patient's condition is normal before the test is performed, Prob_{Dis} is the probability that the patient has the disease before the test is performed, C_{FP} is the cost (i.e. the financial cost and/or health “cost”) of a false-positive result, C_{TN} is the cost of a true-negative result, C_{FN} is the cost of a false-negative result, and C_{TP} is the cost of a true-positive result.

An alternative measure of the accuracy of a diagnostic test is the area under the ROC curve [Zweig, M. H., Campbell, G. Receiver-operating characteristic (ROC) plots: a fundamental evaluation tool in clinical medicine, 1993. Clin. Chem., 39, 561-577] which can take on values between zero and unity. A test with an area under the ROC curve of unity is perfectly accurate because the sensitivity is unity when the FPR is zero, whereas a test with zero area is perfectly inaccurate, meaning that diseased patients are incorrectly given negative test results, while patients without disease are incorrectly given positive test results. The practical lower bound for the ROC curve area is thus 0.5. In FIG. 9, the area under the diagonal line (called the chance diagonal) from 0.0 to 1.1 is 0.5.

Using random guessing to distinguish patients with disease from patients without disease would result in an ROC curve falling along the chance diagonal, hence diagnostic tests with ROC curve areas greater than 0.5 have some discriminatory power. The closer the ROC curve area is to unity, the better the diagnostic test. The area under the ROC curve is interpreted as the probability that a randomly selected patient with disease has a test result that indicates greater suspicion than a randomly chosen patient without disease. Thus, the ROC curve area is a good summary measure of test accuracy because it does not depend on the prevalence of disease or the thresholds used to form the curve.

To summarize, Receiver Operating Characteristics provide meaningful metrics that can be used to indicate the presence/extent of neoplastic growth. Atmospheric air consists of about 20.8% O₂ by volume. The mole fraction of ¹⁶O is nominally 0.99757 with a range of variation 0.99738-0.99726 [Emsley, J., 2001]. According to this reference, the mole fraction of ¹⁸O is much smaller, nominally 0.00205 but can vary between 0.00188-0.00222. By altering the mole fraction of input ¹⁸O, e.g. to 0.5, cancer cells are provided with opportunity to uptake more of this isotope which is detectable in the exhaled breath.

### Properties of Neoplasia Enabling Detection

Literature on cancer reports that cell membrane alterations are a regular feature of neoplasia. The plasma (cellular) membrane controls the movement of substances in and out of cells and is selectively permeable to ions, organic molecules, oxygen molecules and CO₂. The cell membrane is the site-of-origin of alterations fundamental to the initial processes of “malignancy” and to the immunobiology of tumors. In cells transitioning to the neoplastic state, it was suggested [Wallach, D. F. H., Cellular Membranes and Tumor Behavior: A New Hypothesis. Microbiology, 1968. 61: p. 868-874] and confirmed [Provenzale, J. M., S. Mukundan and M. Dewhirst, The Role of Blood-Brain Barrier Permeability in Brain Tumor Imaging and Therapeutics. American Journal of Roentgenology, 2005. 185(3): p. 763-767] that oncogenes introduce inappropriate proteins into cell membranes either in replacement of or in addition to normal components, altering membrane functions. The presence of an abnormal, structural component results in morphological changes [Schultz, W. A., Molecular Biology of Human Cancers An Advanced Student's Textbook 2005, Dordrecht: Springer Science+Business Media, Creekmore, A. L., et. al., Changes in gene expression and cellular architecture in an ovarian cancer progression model. PLoS One, 2011. 6(3): p. e17676] and altered permeability [McCance K. L., Mooney K H, Roberts L K., Pathophysiology, 1990. Excerpt Available From: http://staryweb.fmed.uniba.sk/patfyz/ANGL/cancer3.pdf]. Such disorganization affects the cellular membrane surface [Provenzale, J. M., S. Mukundan and M. Dewhirst, The Role of Blood-Brain Barrier Permeability in Brain Tumor Imaging and Therapeutics. American Journal of Roentgenology, 2005. 185(3): p. 763-767, Anghileri, L. J., M. C. Crone-Escanye, P. Thouvenot, F. Brunotte and J. Robert, Mechanisms of Gallium-67 accumulation by Tumors: Role of Cell Membrane Permeability. J. Nucl. Med, 1988. 29: p. 663-668, LeBreton, E., Mouli, Y., Biochemistry and Physiology of the Cancer Cell. 1988, 29, p. 663-668]. Pathogenesis occurs in mitochondria and contributes to oncogenesis and cancer progression through disabling apoptosis. Proteomic studies utilizing ¹⁸O₂ and ¹⁶O₂ ratios as tracers [Chi, L. M., C. W. Lee, K. P. Chang, S. P. Hao, H. M. Lee, Y. Liang, C. Hsuch, C. J. Yu, I. N. Lee, Y. J. Chang, S. Y. Lee, Y. M. Yeh, Y. S. Chang, K. Y. Chien, J. S. Yu, Enhanced Interferon Signalling Pathway in Oral Cancer Revealed by Quantitative Proteome Analysis of Microdissected Specimens using 16O/18O Labeling and Integrated 2-Dimensional LC-ESI-MALDI Tandem MS. Mol. Cell Proteomics, 2009. 7: p. 1453-1474; Zang, L., D. P. Toy, W. S. Hancock, D. C. Sgroi, B. L. Karger, Proteomic Analysis of Ductal Carcinoma of the Breast Using Laser, Capture Microdissection, LC-MS, and 16O/18O Isotopic Labeling. Journal of Proteome Research, 2004. 3: p. 604-612; Cottingham, K., 16O/18O Labeling in the Spotlight. Journal of Proteome Research, 2004. 3(3): p. 1] identified the presence of altered proteins affecting mitochondrial functions.

Vascular permeability is essential for the health of normal tissues and is also an important characteristic of acute inflammation and pathologies associated with angiogenesis such as tumors, wounds and chronic inflammatory diseases in which permeability is greatly increased (see [Nagy, A. J., B., Laura, Z., Huiyan, Dvorak, M. A., Dvorak, F. H. Vascular permeability, vascular hyperpermeability and angiogenesis. Angiogenesis, 2008, 11, p. 109-119] and the references therein). As a result, tumor progression is associated with disorganized angiogenesis, which results in inadequate oxygen supply. Chronic vascular hyperpermeability characterizes pathological angiogenesis [Nagy, A. J., B., Laura, Z., Huiyan, Dvorak, M. A., Dvorak, F. H. 2008].

The exchange of small molecules (diffusion) is driven by the molecular concentration gradient across the vascular endothelium as determined by Fick's law. The diffusion rate (e.g., ml/s) of a solute depends on the diffusion coefficient for that solute, the surface area available for exchange, the thickness of the capillary and the concentration gradient between the plasma and the interstitial fluid. Changes in any of these parameters will alter the diffusion rate. According to [Nagy, A. J., B., Laura, Z., Huiyan, Dvorak, M. A., Dvorak, F. H. 2008], the permeability is related to the diffusion coefficient, which is radically modified when cells are transitioning to the cancerous state. Thus, quantifying the relationship between tumor oxygenation and vascular functions has a profound impact on preclinical and clinical cancer research. The references underscore four germane facts: (1) vasculature is greatly increased in the presence of cancer, causing alterations in the (oxygen) diffusion coefficient, (2) the plasma membrane is morphologically altered (3) biomarkers function by isotopic substitutions and (4) H₂O freely equilibrates with all compartments in biological systems. These changes in the cell membrane transitioning to the neoplastic stage subsequently cause changes to the membrane permeability of ¹⁶O₂ relative to ¹⁸O₂. Vascular hyperpermeability and angiogenesis are directly correlated with the development of cancer in the body. Furthermore, oxygen isotope fractionation results from various metabolic processes. Differing oxygen isotope ratios can also result from smoking, exercise and disease. For example, there is increased fractionation in smokers due to their compromised ability to diffuse oxygen through the pulmonary membranes. People who engage in routine exercise exhibit a decrease in fractionation because of the increased rate of respiration. It is established that oxygen isotope fractionation is lower in people suffering from disease (e.g. anemia) as the binding of oxygen to hemoglobin is a fractioning process [Reitsema, J. L., Crews, D. E., Oxygen Isotopes as a Biomarker for Sickle-Cell Disease? Results from Transgenic Mice expressing Hemoglobin s. Genes. American Journal of Physical Anthropology, 2011, 145, p. 495-498]. In addition, animal studies involving the bone tissue of sick mice showed a significantly lower isotope ratio than those of the healthy control (−5.6‰ o vs. −4.5‰) mice. In fact, the sickest mice had the lowest isotope ratios −5.8‰ [Reitsema, J. L., Crews, D. E. 2011]. It is expected that cancer cell membranes exhibit an anomalous permeability to heavier isotopes, i.e. discriminate less between ¹⁶O₂ vs. ¹⁸O₂. The method of the present invention proceeds as follows: a volume of premixed air, e.g. ˜80% N₂, 10% ¹⁶O₂ and 10% ¹⁸O₂ (i.e. ¹⁸O₂ is now 240 times the normal concentration) is inhaled. Upon entering the lungs, the O₂ isotopologues bind to hemoglobin and are transported by the blood to all parts of the body where they are used for cellular metabolism. Nearly all of the innocuous ¹⁸O₂ contained in the premixed air is removed from the inhalant (i.e. is consumed during metabolism). Deoxyhemoglobin then transports the ¹⁸O₂ to the lungs where it is exhaled in the form of CO₂. The ratio of ¹⁸O₂ to ¹⁶O₂ is altered relative to what was inhaled and departures from the norm in this ratio are indicative of the presence and/or proliferation of cancer cells.

Although the method of the present invention is applicable for detecting different types of cancer (there are 200 different types of cancer), it is most suitable for the detection of lung cancer, (adenocarcinomic human alveolar basal epithelial). Lung cancer is the most commonly occurring cancer in the general population with over 1.3 million cases worldwide [http://www.who.int/mediacentre/factsheets/fs297/en/]. In addition, early stage diagnosis (within the first five years) can produce a 30%-80% survival rate, hence, the need for early detection is crucial. Inhaled air pervades the lungs. Consequently, impairment of alveoli function from cancer makes the respiratory system ideally suited for the application of the photoacoustic spectroscopic breath analyzer. The alveoli are unique structures in that they are the end points for the capillaries. Alveoli permit the exchange of oxygen in the blood by diffusion. Because of their function, the presence of epithelial cells transitioning to the cancerous state are best identified at this location from the isotopic ratio effect.

Because of its similarities to human anatomy and physiology, particularly with respect to the cardiovascular, urogenital, nervous and musculoskeletal systems, the dog has a long history as a model in drug discovery and development research [Shearin, A. L. and E. A. Ostrander, Leading the way: canine models of genomics and disease. Disease Models & Mechanisms, 2010. 3(1-2): p. 27-34]. Dogs also develop cancers that share many characteristics with human malignancies [Khanna, C., K. Lindelblad-Toh, D. Vail, C. London, P. Bergman, L. Barber, M. Breen, B. Kitchell, E. McNeil, J. F. Modiano, S. Niemi, K. E. Comstock, E. Ostrander, S. Westmoreland, S. Withrow, Dog as a Cancer Model. Nature Biotechnology, 2006. 24(9): p. 1065-1066]. Dog cancers mimic the development of cancers in humans by exhibiting tumor growth over long periods in the setting of an intact immune system, inter-individual and intra-tumor heterogeneity, the development of recurrent or resistant disease and metastasis to relevant distant sites. Other strong similarities of dog to human cancers include histological appearance, tumor genetics, biological behavior and response to conventional therapies. For these reasons, the method of the present invention is also directly applicable to canine cancer treatment monitoring using appropriate enclosures (e.g. oxygen cages) to administer combinations of ¹⁶O₂ and ¹⁸O₂.

I claim:

1. A method for detecting the presence of cancer cells in an animal comprising administering a measured volume of gas comprising air or a mixture of gases wherein measured amounts of ¹⁶O₂ and ¹⁸O₂ are present and then measuring the δ¹⁸O in the CO₂ of the patient's exhaled breath.

2. A method according to claim 1, wherein the animal is a human or other mammal.

3. A method according to claim 1, wherein an increase of δ¹⁸O indicates the presence and stage of cancer.

4. A method according to claim 1, wherein a premixed volume of three gases (¹⁶O₂ and ¹⁸O₂ and ¹⁴N₂) is applied in variable concentrations by volume.

5. A method according to claim 1, wherein δ¹⁸O in exhaled CO₂ is determined by measurement of mid-infrared absorption using at least one mid-infrared laser source.

6. A method according to claim 1, wherein δ¹⁸O in exhaled CO₂ is determined by measurement of mid-infrared absorption using a photoacoustic spectrometer detection system comprising at least one mid-infrared laser source.

7. A method according to claim 6, wherein the results of the analysis are presented on a visual display.

8. A method according to claim 6, wherein the photoacoustic spectrometer detection system also comprises two photoacoustic cells, an airflow system with air filters, a computer and a visual display.

9. A method according to claim 6, wherein the photoacoustic system is comprised of (1) at least one quantum cascade laser, (2) a device for periodically blocking the light emitted by said laser, (3) sample and reference cell compartments, (4) a cantilever acoustic transducer, (5) a beam splitter that directs the intermittent laser light to the reference cell and also redirects the intermittent laser light onto a mirror which illuminates the sample cell and (6) a Michelson-type interferometer.

10. A method according to claim 6, wherein the gas concentrations in the patient's exhaled CO₂ flowing through the photoacoustic spectrometer system are measured simultaneously at wave numbers 2312.85 cm⁻¹ and 2313.15 cm⁻¹.

11. A method according to claim 6, wherein the fractional sensitivity of the photoacoustic spectrometer system, in terms of the minimum discernible changes in δ¹⁸O, is at least 8.3×10⁻².

12. A method according to claim 6, wherein the animal's exhaled CO₂ is analyzed by the photoacoustic spectrometer system to obtain δ¹⁸O, Said δ¹⁸O is visually displayed on an appropriate visual device, and the presence and stage of cancer cells is indicated as increasing differences in the δ¹⁸O averages between populations of healthy individuals and individuals afflicted with cancer. Receiver operating characteristics are employed to evaluate the level of significance of test thresholds used to demarcate afflicted from normal individuals.

13. A method according to claim 6, wherein the pressure of the gas in the photoacoustic spectrometer system is maintained at 8 kPa.

14. A photoacoustic spectrometer system for detecting the presence of cancer cells in an animal comprising an administration device for administering a measured volume of gas comprising air or a mixture of gases wherein measured amounts of ¹⁶O₂ and ¹⁸O₂ are present and detectors are employed for measuring the δ¹⁸O in the CO₂ of the patient's exhaled breath.

15. A photoacoustic spectrometer system according to claim 14, wherein said animal is a human or other mammal, comprising a breathing apparatus for human patients or for other mammals, wherein the breathing apparatus for humans comprises a mask connected to a flow system and the breathing apparatus for other mammals comprises a tent connected to a flow system.

16. A system according to claim 14, wherein the animal is a human or other mammal.

17. A system according to claim 14, comprising at least one mid-infrared laser source capable of producing measurements of δ¹⁸O in exhaled CO₂.

18. A system according to claim 14, which comprises at least mid-infrared laser source and wherein δ¹⁸O in exhaled CO₂ is determined by measurement of mid-infrared absorption using two photoacoustic cells.

19. A system according to claim 14, wherein the photoacoustic spectrometer detection system also comprises two photoacoustic cells, an airflow system with air filters, a computer and a visual display or a recording device.

20. A system according to claim 14, comprising two photoacoustic subsystems wherein each photoacoustic subsystem is a comprised of (1) at least one quantum cascade laser, (2) a device for periodically blocking the light emitted by said laser, (3) test and reference cell compartments, (4) a cantilever acoustic transducer, (5) a beam splitter that directs the intermittent laser light to the reference cell and also redirects the intermittent laser light onto a mirror which illuminates the test cell and (6) a Michelson-type interferometer.

21. A system according to claim 14, wherein the gas concentrations in the patient's exhaled CO₂ flowing through the photoacoustic spectrometer system can be measured simultaneously at wave numbers 2312.85 cm⁻¹ and 2313.15 cm⁻¹.

22. A system according to claim 14, wherein the fractional sensitivity of the photoacoustic spectrometer in terms of the minimum discernible changes in δ¹⁸O, is at least 8.3×10⁻².

23. A system according to claim 14, comprising means for analyzing the animal's exhaled CO₂ to obtain δ¹⁸O, a visual display for displaying said δ¹⁶O or a recording device for recording said δ¹⁸O.

24. A system according to claim 14, comprising a pressure regulating device for maintaining the pressure of the gas in the photoacoustic spectrometer system at 8 kPa.

25. A system according to claim 14, wherein the photoacoustic spectrometer system comprises (1) two quantum cascade lasers, one of said lasers tuned to 2312.85 cm⁻¹ and the other of said lasers tuned to 2313.15 cm⁻¹, (2) two photoacoustic modules, each module employing one cantilever transducer, wherein said two photoacoustic modules operate independently and are used to measure the concentrations of ¹⁸O₂ and ¹⁶O₂ in the exhaled CO₂, (3) two Michelson-type interferometers, (4) two beam splitters that are used to illuminate the reference and sample cell of each photoacoustic cell, (5) two stepper motors connected to modulating shutters, (6) two mirrors, (7) a computer, wherein the computer utilizes software to calculate concentrations of ¹⁸O₂ and ¹⁶O₂ in the exhaled CO₂ from the measured interferometer signals and to compute δ¹⁸O, (8) an air flow control system, (9) a breathing apparatus for human patients or for other mammals, wherein the breathing apparatus for humans comprises a mask connected to a flow system and the breathing apparatus for other mammals comprises a tent connected to a flow system, and (10) a premixed gas supply comprising ¹⁸O and ¹⁶O.

26. A system according to claim 14, wherein the fractional sensitivity is, in terms of the minimum discernible changes in δ¹⁸O, at least 8.3×10⁻².
